(19) United States
(12) Patent Application Publication
(10) Pub. No.: US 2024/0074478 A1
(43) Pub. Date: Mar. 7, 2024

(54) COMPOSITIONS INCLUDING FILAMENTOUS FUNGAL BIOMASS AND CULTURED ANIMAL CELLS, AND METHODS OF FORMING AND USING

(71) Applicant: The Regents of the University of California, Oakland, CA (US)

(72) Inventors: David BLOCK, Oakland, CA (US); Minami OGAWA, Oakland, CA (US)

(21) Appl. No.: 18/504,646

(22) Filed: Nov. 8, 2023

Related U.S. Application Data
(63) Continuation of application No. PCT/US2022/028637, filed on May 10, 2022.
(60) Provisional application No. 63/187,178, filed on May 11, 2021.

(51) Int. Cl. A23L 31/00; A23L 13/40; A23L 17/00; C12N 1/14; C12N 5/071

(52) U.S. Cl. CPC A23L 31/00; A23L 13/424; A23L 17/65; C12N 1/14; C12N 5/0602; C12R 2001/66

(57) ABSTRACT

Set forth herein is a composition that includes a fungal pellet and animal cells, wherein the animal cells are in connection with the fungal pellet. The animal cells can be located on the exterior surface of the fungal pellet, the interior of the fungal pellet, or both. The fungal pellet can include intact fungal cells. The fungal pellet can be substantially inviable. In an example, the pellet can be heat-treated, chemically treated, or lyophilized. The animal cells can be mammalian cells, wherein the mammalian cells may be non-human mammalian cells.

### CROSS REFERENCE TO RELATED APPLICATIONS

This application claims priority to U.S. Provisional Patent Application No. 63/187,178, filed May 11, 2021, which is incorporated by reference herein for all purposes in its entirety.

This invention was made with government support under grant number NSF 2021132 awarded by the National Science Foundation (NSF). The government has certain rights in the invention.

### BACKGROUND

### Field

The rapidly growing global population and limited resources of our bioeconomy highlights the need to find alternative and sustainable methods for the production of food products. Cultivated meat production arises as a potential solution that aims to support these immediate and large scale societal needs. However, there are technical and cost limitations to large scale production due to bioprocessing complexity. Existing technologies use planar culture systems, which are limited and not scalable because of their low surface area to volume ratio and lack of pH, gas and metabolite concentration control. More information can be found in Derakhti S, Safiabadi-Tali S H, Amoabediny G, Sheikhpour M. Attachment and detachment strategies in microcarrier-based cell culture technology: A comprehensive review. Mater Sci Eng C Mater Biol Appl. 2019 October; 103:109782; and Oh S K W, Chen A K, Mok Y, Chen X, Lim U-M, Chin A, et al. Long-term microcarrier suspension cultures of human embryonic stem cells. Stem Cell Res. 2009 May; 2(3):219-30. Information with regard to the number of cells needed for meat production can be found in Rowley J, Abraham E, Campbell A, Brandwein H, Oh S. Meeting Lot-Size Challenges of Manufacturing Adherent Cells for Therapy. J BioProcess Int. 2012; and Moritz M S M, Verbruggen S E L, Post M J. Alternatives for large-scale production of cultured beef: A review. J Integr Agric. 2015 February; 14(2):208-16.

Edible microcarriers have risen as a possible technique to achieve scalable meat production while completely eliminating dissociation and degradation steps necessary when using non-edible microcarriers. However, there is currently little information on the available edible biomaterials that support muscle stem cell growth for meat production since current microcarriers rely on borrowed technology optimized for use in the medical field and not for human consumption.

### SUMMARY OF THE INVENTION

In an example, the disclosure includes a composition including a fungal pellet and animal cells, wherein the animal cells are in connection with the fungal pellet, wherein the fungal pellet is inviable. The animal cells can be located on the exterior surface of the fungal pellet, the interior of the fungal pellet, or both. The fungal pellet can include intact fungal cells. The fungal pellet can comprise filamentous fungus. The fungal pellet can be substantially inviable. In an example, the pellet can be heat-treated, chemically treated, or lyophilized. The fungal pellet can include fungus selected from Rhizopus, Aspergillus, and Penicillium. The fungal pellet can include fungus selected from Rhizopus oligosporus, Aspergillus sojae, Aspergillus awamori, Aspergillus oryzae, Aspergillus tubingensis, Aspergillus nishimurae, and Penicillium chrysogenum. The eukaryotic cells can be mammalian cells, wherein the mammalian cells may be non-human mammalian cells. The animal cells can be fish cells. The eukaryotic cells can be viable. In an example, the composition may be edible. The fungal pellet can have a diameter of 0.8 mm to 1.1 mm. The volume of the fungal pellet can be between 0.013 mm³ to 0.018 mm³. The disclosure includes a food product of the disclosed composition.

In an example, the disclosure includes a method for producing a composition of animal cells including obtaining a fungal pellet comprising a fungus, wherein the fungus is inviable or inactivated, seeding a plurality of animal cells onto the fungal pellet to produce a seeded fungal pellet; and incubating the seeded fungal pellet under conditions whereby the animal cells remain viable. The method may further include harvesting the seeded fungal pellet. In an example, the fungal pellet is obtained by inoculating fungal spores into a liquid medium, whereby after a period of incubation, the fungal spores form a fungal pellet comprises of mycelium. In an example, the fungus is inviable or inactivated by heat-treating or chemically treating the fungal pellet. The animal cells can be mammalian cells such as cow, pig, chicken, goat, and/or sheep. The animal cells can be fish cells. The fungal pellet can obtained by inoculating fungal spores into a liquid growth medium, wherein after an incubation period of at least 2 hours at a temperature between 15° C. to 45° C., the fungal spores form a fungal pellet comprising mycelium. The fungal pellet can include filamentous fungus.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list. Thus, the scope of the present disclosure should not be limited to the specific illustrative structures described herein, but rather extends at least to the structures described by the language of the embodiments, and the equivalents of those structures. Any of the elements that are positively recited in this specification as alternatives may be explicitly included in the embodiments or excluded from the embodiments, in any combination as desired. Although various theories and possible mechanisms may have been discussed herein, in no event should such discussions serve to limit the patentable subject matter.

### DESCRIPTION OF FIGURES

Embodiments of the invention are further described hereinafter with reference to the accompanying drawings, in which:

FIG. 1 illustrates a growth cure of the fungal pellets according to Example 1.

FIG. 2 is a collection of scanning electron microscopy (SEM) images of whole pellets, inner and outer surfaces of the AFP, HFP, and CPF of Example 1.

FIG. 3 is a collection of SEM images stained with Nile Red according to Example 1.

FIG. 4 is a collection of SEM images of AYB, HYB, and CYB of Example 2.

FIG. 5 is a flow chart of an example of a method disclosed herein according to Example 3.

FIG. 6 illustrates a collection of brightfield, red fluorescent protein and composite images according to Example 3.

FIG. 7 is a bar graph of cell viability assessed by the AlamarBlue™ Cell Viability Assay according to Example 3.

FIG. 8 is a collection of images using ImageXpress™ Pico Automated Cell Imaging System according to Example 4.

FIG. 9 is a bar graph of cell viability assessed by the AlamarBlue™ Cell Viability Assay according to Example 4.

FIG. 10 is a flow chart of an example of a method disclosed herein according to Example 6.

FIG. 11 is a bar graph of cell viability assessed by the AlamarBlue™ Cell Viability Assay according to Example 6.

FIG. 12 is a bar graph depicting the hydrophobicity index according to Example 7.

FIG. 13 is a bar graph of cell viability assessed by the AlamarBlue™ Cell Viability Assay according to Example 10, wherein images of the corresponding HFP fungal microcarriers after 3 days of cultivation are shown.

FIG. 14 is a graph of AlamarBlue™ absorbance difference as a function of time according to Example 9.

FIG. 15 is a flow chart of a method disclosed herein according to Example 10.

FIGS. 16A-16C are images of the no microcarrier, cytodex, and fungal pellets, respectively, according to Example 10. FIG. 16D is a bar graph of the viability of the cells according to Example 10.

FIG. 17 is a flow chart of a method disclosed herein according to Example 11.

FIG. 18 is a bar graph of the viability of the cells according to Example 11.

FIG. 19 is western blot showing desmin expression in differentiated C2C12 cells according to Example 12.

### DETAILED DESCRIPTION OF ILLUSTRATED EMBODIMENTS

To address the scalability bottleneck, microcarriers can be used to achieve higher cell densities. Microcarriers are physical spheres that provide a surface area to which cells that require an anchor can adhere and proliferate. They remain suspended in the medium, providing a three dimensional culture environment while offering a large surface area/volume ratio.

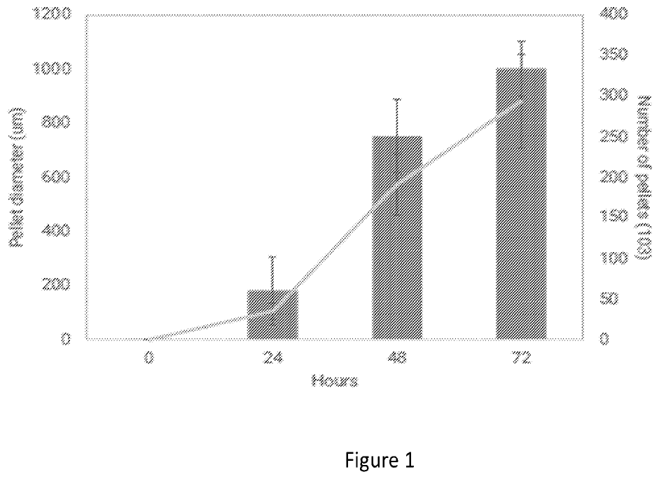

Microcarriers can be made of various materials. For the purpose of cultured meat, edible microcarriers that can be incorporated into the final meat product may be the most promising because their use may eliminate steps necessary to dissociate or dissolve the microcarriers, which can be a challenge when working with non-edible microcarriers (see for example FIG. 1). Such edible microcarriers may be necessary or at the very least desirable for the eventual scale up and optimization for cultured meat production.

Disclosed herein are compositions including 3-dimensional filamentous fungal biomass; and cultured eukaryotic cells, such as animal cells, wherein the filamentous fungal biomass provide a 3-dimensional surface for growth of the cultured eukaryotic cells. Methods herein include growing filamentous fungus into a 3-dimensional shape, such as a pellet; inactivating the filamentous fungus; adding animal cells to the inactivated filamentous fungus; growing the animal cells on the inactivated filamentous fungus. Also provided herein are compositions comprising cultured animal cells, cultured in combination with filamentous fungal biomass.

The filamentous fungi biomass is utilized as a platform on which eukaryotic cells such as animal cells, for example, muscle cells can attach, proliferate, and differentiate. Specifically, edible filamentous fungi spheres (also referred to herein as fungal pellets, filamentous fungal pellets and filamentous fungus biomass) are disclosed herein as a cost effective, alternative microcarrier for the purpose of culturing cells, such as culturing animal cells for cultured meat production. The interaction of the filamentous fungi with the cells are presented, its scalability tested, and its effect on organoleptic properties of the final product evaluated.

Inactive filamentous fungi matrix may be an edible biomaterial for microcarriers to be used in cellular agriculture. While the matrix of filamentous fungi is structurally complex, it is abundant and malleable, providing a rich platform on which we can attach cells. Information obtained from evaluating filamentous fungus mycelium as a novel biomaterial promotes further understanding of microcarrier/muscle cell interaction and serves as a foundation of knowledge and technology to develop other material sources for cellular agriculture. Furthermore, this novel technology improves scalability and consolidates processes in cultured meat production.

Embodiments disclosed herein include a filamentous fungal microcarrier with animal cells attached thereto. Embodiments disclosed also include inactive filamentous fungal pellets for use as a microcarrier, such inactive biomass has not been previously shown by others in combination with animal cells to support their growth. In embodiment herein, the fungal pellets with cultured animal cells provide food ingredients and food products where the microcarrier (fungal pellet) and the cultured cells (e.g., cultured animal cells) are edible. For cultivated meat production, edible supports that can be included in the final product, facilitates processing.

For example, as shown herein, edible filamentous fungi can form fungal pellets as a microcarrier for animal cells to attach and grow, such as for cultivated meat production. Food grade filamentous fungi, such as Aspergillus oryzae and Fusarium venenatum, can be used in the present compositions and methods. Generally Recognized as Safe (GRAS) are commercially available to consume as an alternative form of meat-like products, known as mycoprotein, since 1985. The mycelium, or the vegetative structure of filamentous fungi, is an ideal material to be used for meat alternative products because it can be easily manipulated organoleptically. Filamentous fungi can be considered an abundant, cost effective, sustainable raw material because it is fast growing and is able to be shaped into different geometries. Filamentous fungal biomass can be constructed of intertwining hyphae aggregates that are porous and allows high mass transfer. Structurally, the hyphae cell wall is composed of layers of mannoproteins, glucans and chitin, which gives filamentous fungus both high-protein and high-fiber nutritional value. While many diverse uses have been investigated, filamentous fungi have not been utilized as the attachment of animal cells in the realm of cultivated meat.

The present disclosure includes filamentous fungal biomass with animal cells attached thereto. The methods disclosed herein can include: growing filamentous fungus in nutrient rich liquid media, inactivating the filamentous fungus, adding animal cells thereto, attaching, growing, differentiating, or some combination thereof, the animal cells on the filamentous fungus in a suspension in liquid media, and harvesting the product of the cells and filamentous fungus. Alternatively, or in addition, the fungal mass can be grown in a variety of shapes and sizes and used as the base for distributing muscle, fat, or connective tissue cells to create structured products.

The disclosure also includes grown animal cells removed from the filamentous fungal microcarrier that the animal cells were grown on. The disclosure further includes a combination of cultured animal cells and the filamentous fungal microcarrier with which they were grown.

Filamentous fungal biomass (fungal pellets), such as inactive filamentous fungal biomass, may be a useful microcarrier for animal cell attachment and growth in comparison to other synthetic substrates, such as synthetic microcarriers, because of any of the following advantages i) the edible nature of the fungal filamentous matrix, ii) the low cost of the fungal filamentous matrix, iii) the fungal filamentous matrix can be easily molded into different geometries, iv) the fungal filamentous matrix is already a consumer accepted food ingredient, v) the fungal filamentous matrix is sustainably culturable, vi) the fungal filamentous matrix is able to produce beneficial metabolites, vii) the fungal filamentous matrix naturally produces extracellular matrix proteins which animal cells use for attachment.

Disclosed herein are compositions and methods including fungal pellets and animal cells. In embodiments, the fungal pellets are formed from filamentous fungi that form pellets. These fungal pellets are associations of the filamentous fungal cells that form in liquid culture. Such fungal pellets are generally aggregates of the cells with intertwining hyphae. In some cases, the fungal pellets are spherical in shape. In some cases, fungal pellets can be cylindrical, tube-like, filament-like or may be irregular in shape. In some cases, fungal pellets have rounded edges. In some cases, fungal pellets have non-rounded or more jagged edges. In some cases, fungal pellets are spherical in shape and the edges are primarily rounded.

In some embodiments herein, one or more filamentous fungus is grown, such as in liquid culture, such that it forms pellets. The fungal pellets are then used as a microcarrier for the cultivation of a second type of cell, such as a eukaryotic cell, such as an animal cell. In embodiments, the eukaryotic cells, e.g., animal cells, attached to the fungal cells of the fungal pellets. In embodiments, the eukaryotic cells, e.g., animal cells, proliferate on the fungal pellets. In embodiments, the animal cells differentiate on the fungal pellets.

Disclosed herein are compositions and methods including fungal pellets and eukaryotic cells, wherein the eukaryotic cells are of a different species from the filamentous fungus forming the pellets. In general, the eukaryotic cells are of a different genus from the filamentous fungus. In some embodiments, the eukaryotic cells are of a different kingdom from the filamentous fungus. In embodiments, the eukaryotic cell is an animal cell. In some embodiments, the eukaryotic cell is a bovine cell, a porcine cell, or an ovine cell. In some embodiments, the eukaryotic cell is a fish cell. In some embodiments, the eukaryotic cell is not a fungal cell. In some embodiments, the eukaryotic cell is not a yeast cell.

In an example, the fungal pellet may include edible filamentous fungi that may be microcarriers for cultured eukaryotic cells, such as cultured animal cells, to be used in food production, such as for cultivated meat. Food grade filamentous fungi, such as Aspergillus oryzae and Fusarium venenatum, are considered Generally Recognized as Safe (GRAS) and have been commercially available to consume on their own as an alternative form of meat-like products, known as mycoprotein. In the embodiments herein, filamentous fungi are employed as edible microcarriers, providing both a surface and a 3-dimensional shape for the growth of eukaryotic cells, such as animal cells, for use as a food product or edible ingredient.

In some embodiments the filamentous fungus used to form the fungal pellets and for use of such fungal pellets as a microcarrier is selected from the group consisting of Aspergillus sp., Penicillium sp., Agaricus sp., Amanita sp., Armillaria sp., Auricularia, Boletus sp., Bovista, Calbovista sp., Calvatia sp., Cantharellus sp., Chlorophyllum sp., Clitocybe sp., Clitopilus sp., Coprinus sp., Cortinarius sp., Craterellus sp., Entoloma sp., Flammulina sp., Fusarium sp., Gomphus sp., Grifola sp., Polypilus sp., Gyromitra sp., Helvella sp., Hericium sp., Hydnum sp., Hygrophorus sp., Lactarius sp., Leccinum sp., Lentinus sp., Lepiota sp., Lepista sp., Lycoperdon sp., Marasmius sp., Morchella sp., Phlogiotis sp., Pholiota sp., Pleurocybella sp., Pleurotus sp., Pluteus sp., Polypilus sp., Polyozellus sp., Polyporus sp., Ramaria sp., Rozites sp., Russula sp., Sparassis sp., Strobilomyces sp., Stropharia sp., Suillus sp., Terfezia sp., Tremella sp., Tricholoma sp., Tuber sp., Volvariella sp., Rhizopus sp, and any combination thereof. In some embodiments, the filamentous fungi microcarrier is a fungus selected from the group consisting of Rhizopus, Aspergillus and Penicillium. For example, the fungal pellet can include one of more of Rhizopus oligosporus, Aspergillus sojae, Aspergillus awamori, Aspergillus oryzae, Aspergillus tubingensis, Aspergillus nishimurae, and Penicillium chrysogenum.

In some embodiments, the filamentous fungus pellet provides a microcarrier for cultured animal cells for use as food product or edible ingredient. In some cases, the food product is a cultivated meat product. In some cases, the food product is a cultivated fish product. The mycelium, or the vegetative structure of filamentous fungus, is an ideal material to be used for food products such as meat alternative products (e.g., cultivated meat) and cultivated fish products because it can be easily manipulated organoleptically. In an example, the fungal pellets are edible, which refers to fungal pellets that are food grade and Generally Recognized as Safe (GRAS) according to sections 201(s) and 409 of the Federal Food, Drug, and Cosmetic Act. Fungal pellets can be considered an abundant, cost effective, sustainable raw material because it is fast growing and can spontaneously form hollow spheres when grown in aqueous solution under agitation.

The fungal pellets have a naturally porous wall that is constructed of intertwining hyphae aggregates that allows high mass transfer. Structurally, the hyphae cell wall is composed of layers of mannoproteins, glucans and chitin, which gives filamentous fungus high-protein and high-fiber nutritional value. In some embodiments herein, food products comprising the filamentous fungus pellet and cultivated cells have a protein content contributed from the filamentous fungus cells in the pellet as well as the cultivated cells, such as cultivated animal cells. In some cases, the fungal pellet has protein content between about 15% to about 50% on a dry weight basis. In some cases, the protein content of the fungal pellet is between 15-20%, 20-25%, 25-30%, 30-35%, 35-40%, 40-45%, 45-50%, or at least 15%, at least 20%, at least 25%, at least 30%, at least 35%, at least 40%, at least 45% or at least 50% on a dry weight basis.

In some embodiments, the filamentous fungus contributes flavor, nutritional value, bio-accessibility, digestibility, or a combination thereof to the edible product made from the fungal pellets and cultivated animal cells. Filamentous fungus in the combination can contribute to PDCAAS (Protein Digestibility Corrected Amino Acid Score) of the composition. PDCAAS measures protein quality as a ratio of the limiting essential amino acid level versus the. level of the essential amino acid in a reference protein, corrected for digestibility. Fungal protein has a PDCAAS value near 1 (1 is the “ideal value”). In some cases, the fungal pellets of the compositions herein contribute a PDCAAS of between 0.9-1.0, 0.93-1.0, 0.95-1.0, or 0.98-1.0. In some cases, the fungal pellets of the compositions can have a PDCAAS that is equal to or greater than the PDCAAS of a vegetable-derived or nut-derived protein such as soy protein, cashew protein or pea protein. In some cases, the fungal pellets of the compositions can have a PDCAAS that is equal to or greater than the PDCAAS of an animal-derived protein, such as protein from beef, pork or fish. In some cases, the fungal pellets of the compositions contribute dietary fiber to the composition, such as polymeric n-acetyl glucosamine (chitin) and beta 1-3 and 1-6 glucans, which can confer health benefits to food products.

In the present disclosure, the structural properties of the natural matrix of filamentous fungus mycelium are utilized as a novel biomaterial for microcarriers in cellular agriculture. The present edible filamentous fungus can spontaneously form pellets, such as spheres, when grown in liquid culture. For the purpose of creating an edible microcarrier, the first desirable characteristic is that the filamentous fungus must be GRAS, and comply with regulations of use as a food ingredient. GRAS filamentous fungus may be selected, for example, from the Phaff Culture Collection and Viticulture and Enology Culture Collection at UC Davis. Growth morphology of filamentous fungus varies across strain and culture conditions. Therefore, the selected filamentous fungus may be cultured in liquid growth media under agitation and edible filamentous fungus may form compact spheres or other fungal pellet shapes. In some embodiments, A. oryzae may be an ideal strain with which to work because it complies with the desirable characteristics above. Liquid media for growth of the filamentous fungi includes media that supports growth of the fungus and the formation of hyphae.

In some embodiments, after production of the fungal pellets, the fungal pellets are treated to render them substantially inviable or inactivated, before using the filamentous fungus pellets as microcarriers, such that the fungus within pellets are no longer capable of growing. In some embodiments, the majority of the filamentous fungus cells contained within the pellets are inviable, for example the pellets contain at least 80% inviable, at least 85% inviable cells, at least 90% inviable cells, at least 95% inviable cells, or substantially all (about 100%) of the cells in the pellet are inviable. Viability of pellets can be determined, for example, by assessing for cell growth, such as by taking a single fungal pellet, incubating for 5 days at 28° C., and assessing growth or no growth on YPD plates. The fungal pellets can be rendered substantially inviable by being heat-treated, chemically treated, or lyophilized. For example, the fungal pellets may be ethanol treated to inactivate them and ensure that the filamentous fungus acts as an inert support to which the cells can attach. The fungal pellet can be treated with 70% ethanol for 15-30 minutes. In an example, the fungal pellet can be heat treated for 15-30 minutes at a temperature between 110-130° C. The fungal pellet can be lyophilized, for example by lyophilization −42° C. for 48 hours at 80 Pa. The inviable (inactivated) fungal pellets can retain its 3-dimensional shape, providing a 3-dimensional microcarrier for cultivating eukaryotic cells, such as for cultivating animal cells. In an example, the viable animal cells are attached to the inviable fungal cells.

Microcarriers are typically categorized and distinguished based on size, charge, and surface (such as porosity and texture). The gross morphology of the fungal pellets can be characterized by using methods such as scanning electron microscopy and measuring their zeta potential. In some embodiments, desirable sizes of the pellet pellets may be within the range of from 1500 μm to 3000 micrometers (μm) in diameter. In some cases, the fungal pellet diameter is between about 0.5 mm to about 40 cm. In some cases, the fungal pellet has a volume between about 0.02 mm³ to about 34000 cm³. The size of microcarrier has been reported to increase cell attachment compared to smaller microcarriers (500 μm). The larger size may create a surface more similar to a planar surface, that may be ideal because cells fail to develop their normal morphology and multiply on sharply curved surfaces. Similarly, charge and surface properties can be modified or selected using the Taguchi method to improve the fungal pellet microcarriers for cell attachment as discussed below.

Scanning electron microscopy of the microcarriers illustrate bridge formation and bead-to-bead transfer of cells between neighboring microcarriers. These phenomena simplify the process of cell culturing because the growth surface can be expanded simply by adding new microcarriers to the medium. It has also been shown that this mechanism of growth can promote myotubes to express adult muscle characteristics.

Another important characteristic of the microcarrier surface may be the existence of extracellular matrix (ECM) proteins such as collagen, laminin, fibronectin or vitronectin, which may be necessary for integrins to bind. Fungal receptors have a high affinity for ECM proteins, even on hyphae surfaces. Coating an ECM, may be successful on filamentous fungus spheres and ensure cell attachment. These ECM proteins can be quantified by Western immunoblotting.

Non-edible microcarriers such as SYNTHEMAX®, CELLBIND® and CYTODEX®, are effective in bovine myoblast proliferation and differentiation, and can be used as controls. Bovine myoblasts from fresh meat or biopsies of animals can be used or isolated respectively and commercial media can be used.

In some embodiments, the cells cultivated in concert with filamentous fungus pellets are cultivated in a liquid medium. Liquid media for growth of the cultivated cells (the cells seeded on the fungal pellet microcarrier) include liquid media for growth of animal cells, and can include liquid media with components suitable for use in edible products. In some cases, the cultivated cells are animal cells and the liquid media for the animal cells seeded on the fungal pellet microcarrier is a serum-free media.

Proliferation, differentiation and other features of the cultivated cells can be assessed. Proliferation of cells can be measured by quantifying total DNA (Quant-iT PicoGreen kit). Alternatively, viable cells can be measured by assessing cell metabolomic activity using MTT assay. For differentiation, such as for differentiation of myoblasts to myotubes, expression of the early differentiation markers MyoD and Myogenin, and DES (an early stage myogenin marker) can be quantified using quantitative PCR reactions, or other gene expression assay or by protein expression (such as by western blotting). In each of these assays, the values of unseeded microcarriers to account for the filamentous fungus microcarrier can be measured as controls or for background signal. Microscopy and other visualization techniques can be used to assess the size, shape and other hallmarks of differentiation in the cultivated cells.

In some embodiments, the filamentous fungus is cultivated in a liquid medium to form the fungal pellets. For example, cultivation can use flasks, such as 250 ml spinner flasks. Fungal pellet microcarriers can be translated to batch process bioreactors and the effect of the microcarrier on sensory properties assessed. Scaling up can introduce effects such as shear force from media agitation and oxygen availability, factors that are not as significant in spinner flasks. Thus, it may be necessary to adjust variables such as seeding conditions (inoculum and operating parameters), temperature, and nutrient availability amongst others.

Since a unique feature of microcarriers in embodiments herein is that they become an integral part of the final product and contribute to the sensory properties of the product. A descriptive analysis may be performed and the texture and aroma of the product can be evaluated using a Texture Analyzer, Gas Chromatography-Mass Spectrophotometry (GC/MS), and High Performance Liquid Chromatography (HPLC) for example. This information may serve as preliminary data to further improve the fungal pellet microcarriers, characterize food products, and help communicate this novel food product to future consumers.

In some embodiments, the filamentous fungus microcarrier can support or promote proliferation and/or differentiation of the cultivated cells, such as bovine myoblasts. In some embodiments, the filamentous fungus microcarrier, supports or promotes cell viability of the cultivated cells. In some cases, cell viability of the cultivated cells may be improved as compared to no microcarrier and/or as compared to a synthetic microcarrier (e.g., cytodex). In some cases, cell viability of the cultivated cells may be similar to or substantially equivalent as compared to cells cultivated with a synthetic microcarrier (e.g., cytodex). In some cases, cell viability of the cultivated cells may be similar to or substantially equivalent as compared to cells cultivated without a microcarrier. A product of this analysis which utilizes fungal pellet microcarriers may be comparable or have enhanced organoleptic properties compared to cultivated meat made with non-edible microcarriers or made without a microcarrier.

The present composition can include cultivated eukaryotic cells and the filamentous fungus pellet, wherein the cultivated eukaryotic cells are not derived from the same species as the fungal pellet, such as cultivated animal cells. In some embodiments, the cultivated eukaryotic cells (e.g. animal cells) or portion thereof can be located on the exterior surface of the filamentous fungus pellet. In some embodiments, the cultivated eukaryotic cells (e.g., animal cells) or portion thereof are located in the interior of the filamentous fungus pellet. In some cases, the cultivated eukaryotic cells (e.g., animal cells) can be located on both the exterior and the interior of the filamentous fungus pellet. The cultivated eukaryotic cells can be animal cells, mammalian cells, non-human mammalian cells, fish cells, bovine cells, pig cells, chicken cells, goat cells, sheep cells, among others.

In an example, the mammalian cells or animal cells cultivated on the fungal pellets can be derived from livestock, poultry, fish, or insects. As used herein, “livestock” comprises any domestic mammal, semi-domestic mammal, captive wild mammal or extinct mammals. Non-limiting examples of non-human mammals and extinct mammals include: antelope, bear, beaver, bison, boar, camel, caribou, cattle, deer, elephant, elk, fox, giraffe, goat, hare, horse, ibex, kangaroo, lion, llama, mammoth, moose, peccary, pig, rabbit, seal, sheep, squirrel, saber tooth tiger, tiger, whale, yak, and zebra, or combinations thereof. In some embodiments, the cultivated animal cells are derived from bird cells. Non-limiting examples of birds include: chicken, duck, emu, goose, grouse, ostrich, pheasant, pigeon, dodo, mao, quail, and turkey, or combinations thereof. In some embodiments, the cultivated animal cells are derived from fishes. Non-limiting examples of fishes include: bass, catfish, carp, cod, eel, flounder, fugu, grouper, haddock, halibut, herring, mackerel, mahi, marlin, orange roughy, perch, pike, pollock, salmon, sardine, shark, snapper, sole, sturgeon, swordfish, tilapia, trout, tuna, whale, and walleye, or combinations thereof. In some embodiments, the cultivated animal cells are derived from invertebrates. Non-limiting examples of invertebrates include: lobster, abalone, crab, shrimp, clams, octopus, oysters, mussels, squid, and sea urchin. In some embodiments, the cultivated animal cells are derived from reptiles. Non-limiting examples of reptiles include: snake, alligator, and turtle. In some embodiments, the cultivated animal cells are derived from amphibians. Non-limiting example of amphibians includes frogs.

In some examples, mammalian cells cultivated on the fungal pellets comprise myoblasts and/or fibroblasts. In some embodiments, the cells comprises myoblasts, fibroblasts and/or fibroblasts progenitor cells. In some embodiments, the cells comprise myoblasts, fibroblasts and adipocytes. In some embodiments, the cells comprise myoblasts, fibroblasts, adipocytes, and/or fibroblast cells comprise myoblasts, fibroblasts and endothelial cells. In some embodiments, the cells comprise myoblasts, fibroblasts and endothelial cells, and/or fibroblast progenitor cells, and/or endothelial progenitor cells. In some embodiments, the cells comprise myoblasts and smooth muscle cells. In some embodiments, the cells comprise myoblasts, smooth muscle cells and endothelial cells. In some embodiments, the cells comprise myoblasts, smooth muscle cells, endothelial cells, and adipocytes. In some embodiments, the cells comprise myoblasts, fibroblasts, endothelial cells, and adipocytes. In some embodiments, the cells comprise myoblasts, fibroblasts, endothelial cells, adipocytes and/or fibroblast progenitor cells, and/or adipocyte progenitor cells, and/or endothelial progenitor cells.

The method of producing the composition can include obtaining a fungal pellet comprising a filamentous fungus, wherein the fungal cells are rendered inviable or inactivated after the fungal pellet is formed; seeding a plurality of animal cells onto the fungal pellet to produce a seeded fungal pellet; and, incubating the seeded fungal pellet under conditions whereby the animal cells remain viable. In some embodiments of the method, the animal cells are incubated under conditions where the cells proliferate. In some embodiments of the method, the animal cells are incubated under conditions where the cells differentiate. In some embodiments, the method may further include harvesting the seeded fungal pellet.

In an example, the composition includes a filamentous fungal biomass; and cultured animal cells, wherein the animal cells are in connection with the filamentous fungal biomass. In some embodiments, the animal cells are associated with cells of the filamentous fungal biomass (i.e., fungal pellet) through protein-protein interactions between proteins present on the cell wall of the fungal cells and proteins on the surface of the animal cells. In some embodiments, the fungal cells associated with the animal cells are inactivated or inviable fungal cells. The cultured animal cells may be cultured in combination with filamentous fungal biomass. The filamentous fungal biomass may be grown in a desired form. The filamentous fungal biomass may be used as a base for a final product that comprises both the filamentous fungal biomass and at least some of the cultured animal cells. The cultured animal cells may be cultured in combination with a nutrient rich liquid media. The cultured animal cells may be cultured in combination with a serum-free liquid media.

In an example, the method includes growing filamentous fungus; inactivating the filamentous fungus; adding animal cells to the inactivated filamentous fungus; growing additional animal cells on the inactivated filamentous fungus; and harvesting at least some of the grown animal cells. The method may include attaching initial animal cells to the filamentous fungal biomass before any additional animal cells are cultured. The method may include affecting one or more conditions to affect the growth, differentiation, or both of the animal cells.

Cell attachment is a critical step in cell interaction with the substrate. Low attachment efficiency can lead to a low expansion yield. Adhesion mechanism of yeast cells and adhesion mechanisms of animal cells differ.

Adhesion in yeast can be self- or non-self. Self-adhesion of vegetative cells is a common property of S. cerevisiae and is also called flocculation. In S. cerevisiae, flocculation is known to be mediated by several genes. Among them, the FLO or flocculation genes encode cell surface proteins, called flocculins, that directly participate in adhesion of cells to each other or other substrates. The FLO gene family can be divided into two groups. The first group—FLO1, FLO5, FLO9, and FLO10—share considerable sequence homology and are sub-telomeric genes that code for proteins responsible for cell to cell adhesion and form aggregates of many cells known as flocs. These lectin-like proteins recognize and bind to α-mannan residues (receptors) of neighboring cells. Calcium ions confer the active conformation of these proteins. Although the active flocculins only exist on flocculent yeast cells, the receptors may be found on non-flocculent cells since the cells walls of Saccharomyces like other fungi are composed of mannans; hence, the prerequisite of flocculation is the presence of the flocculins.

Without being bound by theory, initial formation of yeast biocapsules (yeast attached to fungal pellet) is thought to begin with the hydrophobic attraction of filamentous fungal spores and yeast in liquid medium. The hydrophobins that cover the spore walls are drawn to the hydrophobicity of the flocculins and oxylipin on yeast cell walls and form an agglomeration of yeast cells and filamentous fungal spores. The spores and yeast cells generally have negative charges which causes a repulsion. However, this force is overcome by the hydrophobic interactions. Once germination begins, the spores will lose their melanin and hydrophobin coating and polysaccharides are exposed, leading to salt bridging between polysaccharides on filamentous fungus and yeast cell walls. After germination, hyphal elongation begins and yeast flocculins can bind to α-mannan residues on hyphal cell walls, similar to the mechanism of yeast flocculation. Hydrophobic forces between oxylipins on filamentous fungus and yeast flocculins may contribute to additional attachment.

Animal cell adhesion is mediated by cell adhesion molecules (CAM) binding to substrates on a microcarrier surface. Within the various CAM, the integrin family is the main surface receptor group that regulates cell adherence. The integrins are transmembrane glycoproteins comprised of 19 α- and 8 β-subunits that are expressed in 25 different a/P heterodimeric combinations on the cell surface. Integrins bind to proteins such as collagen, laminin, fibronectin or vitronectin. These extracellular matrix (ECM) proteins are commonly used to coat microcarriers for the purpose of facilitating cell adherence. The ECM proteins are characterized by a specific amino acid sequence RGD (arginine-glycine-aspartate).

Other factors which contribute to cell adhesion are the physicochemical properties of the carrier surface. These include the chemical properties of the microcarrier such as the charge or hydrophilicity and physical properties such as the stiffness, elasticity, size, shape and topography. These factors may positively or negatively affect the cell adhesion. The positive or negative effects are specific to cell lines and differ during the maturity stages of the cell. For example, nanofibrous surfaces have shown better C2C12 cell attachment opposed to completely smooth surfaces. Smooth surfaces however, may promote differentiation. Speculated reasons of this phenomenon is explained by the fact that the nanofibers more closely mimic ECM surfaces.

In embodiments herein, animal cells are seeded onto fungal pellets and the animal cells attach to cells of the fungal pellet. In some embodiments, fungal cells within the fungal pellet are inactivated or inviable and the animal cells attach to such inactivated/inviable fungal cells. The fungal cell wall contains proteins, that by in silico analysis, contain RGD-like sequences. Without being bound by theory, animal cell integrins may sense and attach to fungal cell wall RGD peptides. In the case of filamentous fungal pellets, the pellet surface is made of cross fibers of hyphal filaments which closely mimic ECM matrix and may lead to increased animal cell attachment to the pellet.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

In this application, terms such as “a,” “an,” and “the” are not intended to refer to only a singular entity but include the general class of which a specific example may be used for illustration. The terms “a,” “an,” and “the” are used interchangeably with the term “at least one.” The phrases “at least one of” and “comprises at least one of” followed by a list refers to any of the items in the list and any combination of two or more items in the list.

As used herein, the term “or” is generally employed in its usual sense including “and/of” unless the content clearly dictates otherwise.

The term “and/or” means one or all of the listed elements or a combination of any two or more of the listed elements.

Also, herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also, herein, the recitations of numerical ranges by endpoints include all numbers subsumed within that range as well as the endpoints (e.g., 1 to 5 includes 1, 1.5, 2, 2.75, 3, 3.80, 4, 5, etc.) and any sub-ranges (e.g., 1 to 5 includes 1 to 4, 1 to 3, 2 to 4, etc.).

As used herein, the term “room temperature” refers to a temperature of 20° C. to 25° C.

The term “in the range” or “within a range” (and similar statements) includes the endpoints of the stated range.

### EXAMPLES

### Example 1: Preparation of Fungal Pellets

Fungal spores of Aspergillus oryzae UCD 76-2 were pre-inoculated onto sporulation media plates (1.7% (w/v) corn meal agar, 0.1% (w/v) yeast extract, 0.2% (w/v) glucose and 2% (w/v) agar) for 7 days at 28 C. Spores were suspended in sterile DI water, the suspension was then vortexed and sonicated for 5 minutes, and 1×10⁶ spore/mL final concentration inoculated in 50 mL of ff pellet medium (6% (w/v) glucose, 0.3% (w/v) yeast extract, 0.3% (w/v) NaNO3, 0.1% (w/v) K₂HPO₄, 0.05% (w/v) MgSO₄, 0.05% (w/v) KCl, 0.001% (w/v) FeSO₄; pH adjusted to 5.5 with HCl) in 250 mL Erlenmeyer flasks covered with hydrophobic cotton. Spores were incubated in the media for 4 days at 30° C. under high agitation (250 rpm), during which time the fungus formed pellets. FIG. 1 shows the growth curve of the fungal pellets; the line represents pellet diameter and grey bars shows the number (×10³) of pellets per flask.

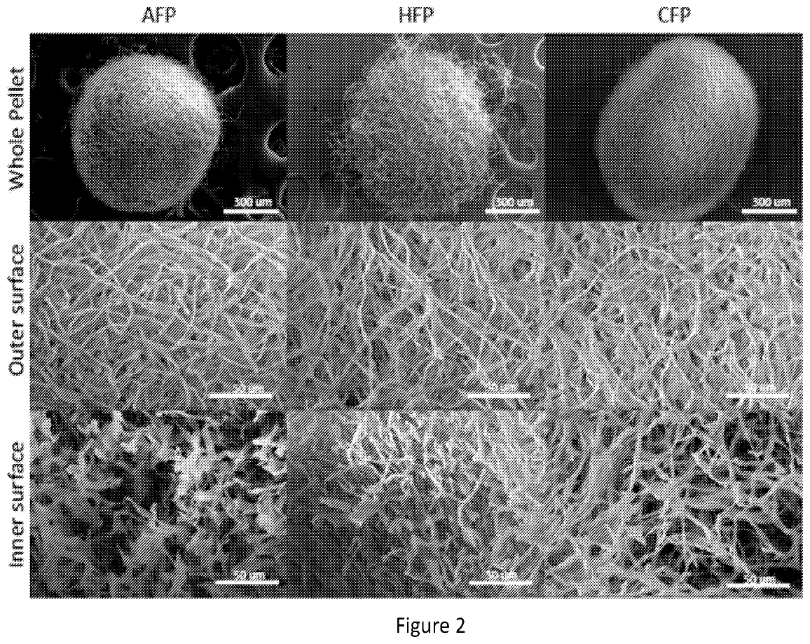

At the completion of the fungal pellet growth phase, the culture was divided into 3 groups: (a) no treatment to retain an active fungal pellet (AFP); (b) heat treating the pellet for 20 minutes at 121° C. (HFP); and (c) chemically treating the pellet with 70% ethanol for 20 minutes (CFP). Viability of pellets was confirmed by taking a single pellet, incubating for 5 days at 28° C. and assessing growth or no growth on YPD plates (Table 1). Each viability check was repeated with 12 randomly chosen pellets. The whole pellets, inner and outer surfaces of the AFP, HFP, and CPF were examined by scanning electron microscopy (SEM). Results are shown in FIG. 2.

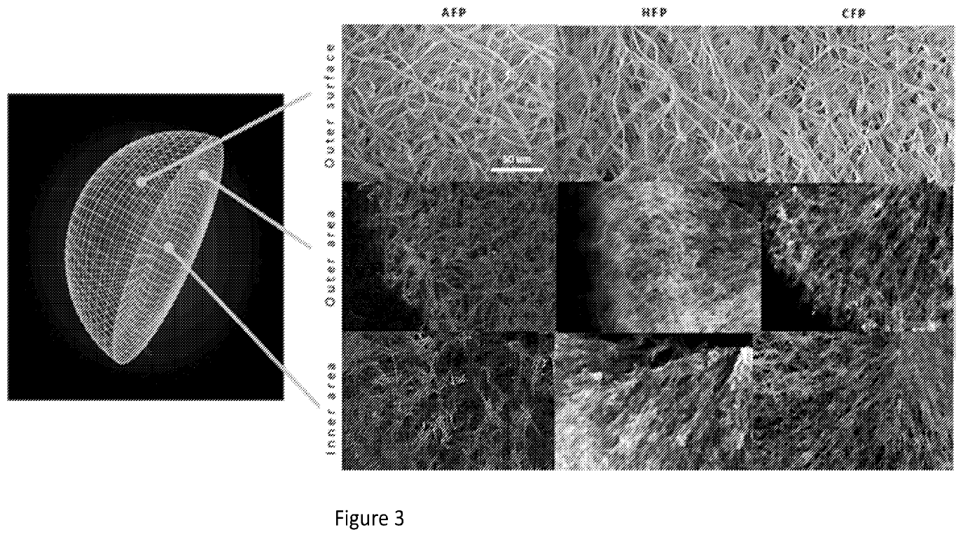

Localized fungal hyphae density of fungal pellets was examined by staining the pellets with Nile Red (FisherScientific) and imaging by confocal microscopy. FIG. 3 shows the outer surface of the pellets taken by SEM, and below, the inner and outer cross sections of pellet stained with Nile Red and imaged by confocal microscopy.

The pellets were further analyzed for physical and chemical characteristics as shown in Table 1. Zeta potential of fungal pellets were taken by Zetasizer (Malvern Panalytical). Pellets were mechanically broken into smaller hyphae fragments using a homogenizer at 14 k setting for 3 min. The mixture was filtered using 300 micron filter to remove larger particles and the remaining suspension was measured for zeta potential. Elemental composition analysis was performed by Thermo Fisher Quattro S Environmental scanning electron morphology equipped with QUANTAX EDS detector. Hydrophobicity of the pellets was measured using a phase-partition method described by Rosenberg et al. (1983). After washing with phosphate buffer saline (PBS), 0.5 ml of settled pellets was placed in 9.5 mL of PBS. Optical density of the cell suspension was measured at 640 nm (A1) using Genesys 10 s UV-Vis spectrophotometer (Thermo Scientific, Rochester, NY, USA). 1.4 ml of octane (Alfa Aesar, Haverhill, MA, USA) was added into the 10 ml of fungal suspension, then mixed with a vortex mixer for 2 minutes and allowed to stand for 10-15 minutes until phase separation was observed. The upper hydrocarbon phase was discarded, and the lower aqueous phase was collected and measured for optical density at 640 nm (A2). The hydrophobicity index (HPBI) was determined as: HPBI=A1−A2/A1*100.

TABLE 1
Fungal Pellet Characteristics

| | | AFP | HFP | CFP |
|---|---|---|---|---|
| Viability (%) | | 100 ± 0 | 0 ± 0 | 17 ± 2 |
| Diameter (mm) | | 0.9 ± 0.1 | 0.97 ± 0.2 | 1.03 ± 0.3 |
| Estimated surface area | | 10.3 ± 2.3 | 9.1 ± 1.5 | 10.3 ± 3.2 |
| (mm\hat{ }2/fungal pellet) | | | | |
| Wet weight (mg/fungal | | 0.37 ± 0.05 | 0.27 ± 0.06 | 0.21 ± 0.04 |
| pellet) | | | | |
| Dry mass (mg/fungal | | 0.04 ± 0.008 | 0.03 ± 0.01 | 0.01 ± 0.004 |
| pellet) | | | | |
| Volume (mm\hat{ }3/fungal | | 0.017 ± 0.005 | 0.014 ± 0.002 | 0.017 ± 0.001 |
| pellet) | | | | |
| Charge or zeta | | −4.3 ± 0.6 | −2.45 ± 0.3 | −2.35 ± 0.2 |
| potential (mV) | | | | |
| Elemental | Carbon | 60.9 ± 10 | 52.8 ± 9 | 49.9 ± 8 |
| composition | % | | | |
| | Oxygen | 39.9 ± 8 | 47.2 ± 8 | 37.3 ± 7 |
| | % | | | |

### Example 2: Yeast Cells Immobilized on Fungal Pellets (Biocapsules)

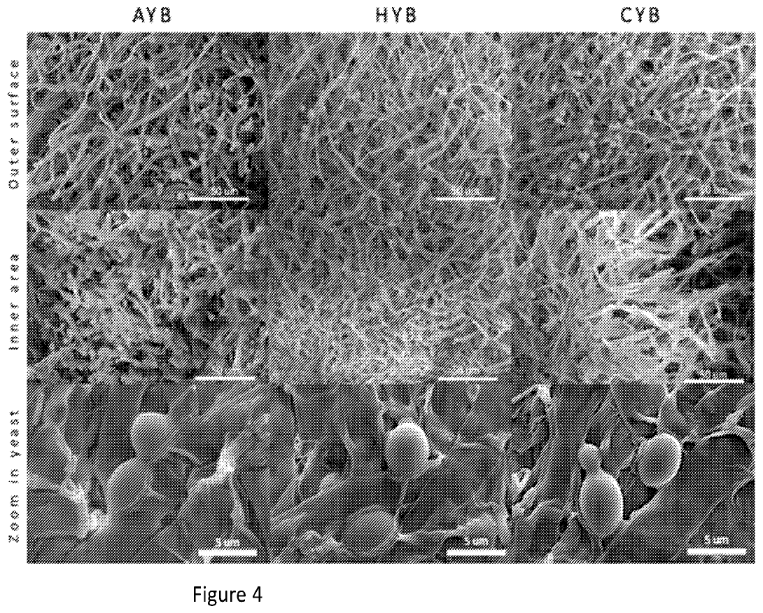

Yeast Saccharomyces cerevisiae G1 were streaked onto YPD agar plates and grown for 3 days at 28° C. Colonies were then inoculated into 50 ml liquid YPD media and grown in a shaker flask for 1 day at 28° C., 150 rpm. The cultures were spun down at 4200 rpm for 10 minutes, supernatant removed and yeasts are weighed and combined with active fungal pellets (AFP) or heat treated fungal pellets (HFP) or chemical treated fungal pellets (CFP) at a ratio of 1:10 in a sterile container. A hundred times the weight of the yeast cells of YPD liquid was added to the same container to submerge pellets and gently shaken to homogenize solution. The container was left loose-capped and yeasts were left to grow overnight at 28° C. At this point, yeasts cells began attaching to fungal mycelium and the yeast-fungus structure is termed yeast biocapsules, which are applied to alcoholic fermentation production such as beer, wine and bioethanol. AFP pellets with yeast are termed active yeast biocapsules (AYB), HFP with yeasts are heat treated yeast biocapsules and CFP with yeasts are chemical treated yeast biocapsules (CYB). After yeast biocapsules incubation had finished, the supernatant was decanted and yeast biocapsules were washed twice with sterile DI water. The pellet-yeast mixtures (yeast biocapsules) were visualized with scanning electron microscopy. Results are shown in FIG. 4. AYB=yeast immobilized on AFP; HYB=yeast immobilized on HFP; CYB=yeast immobilized on CFP. Characterization of the biocapsules is shown in Table 2.

TABLE 2
Immobilized yeast cells on various fungal pellets

| | AYB | HYB | CYB |
|---|---|---|---|
| Total non-immobilized | 344 ± 47 | 410 ± 13 | 407 ± 12 |
| cells (*10\hat{ }6 cells) | | | |
| Total immobilized | 2.5 ± 2.1 | 1.38 ± 0.4 | 4.7 ± 1.9 |
| cells (*10\hat{ }6 cells) | | | |
| Cell immobilization | 56.8 ± 12.3 | 52.0 ± 18.5 | 69.4 ± 32.6 |
| yield (*10\hat{ }6/g WW) | | | |
| Cell immobilization | 0.014 ± 0.001 | 0.015 ± 0.001 | 0.028 ± 0.001 |
| yield (*10\hat{ }6/fungal | | | |
| pellet) | | | |

### Example 3 Immobilization of Animal Cells

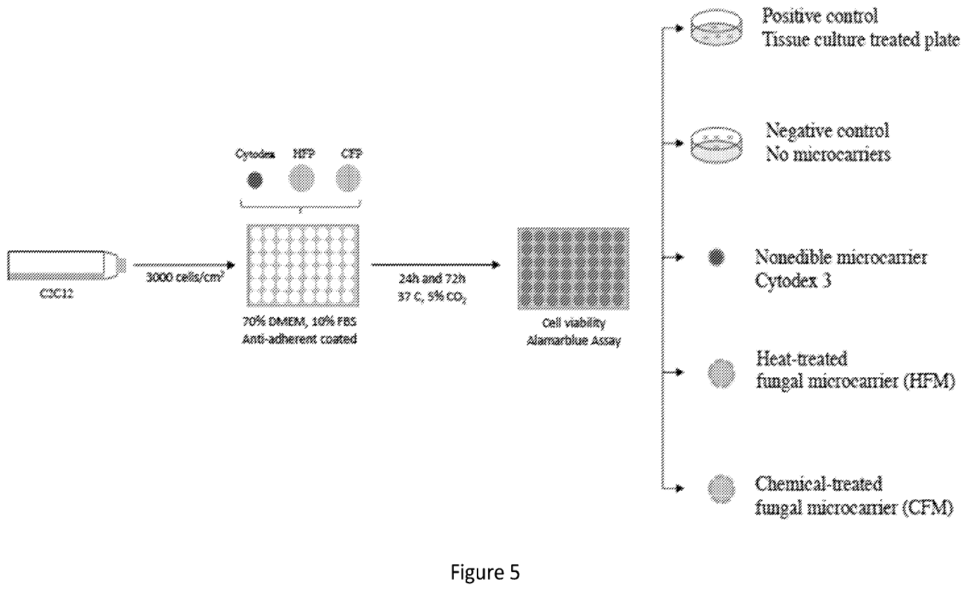

C2C12 cells (mouse myoblasts; ATCC) were seeded at 3000 cells/cm² onto anti-adherent coated (StemCell) multi-well plates (ThermoScientific) in 70% DMEM, media supplemented with 10% fetal bovine serum (FBS). Microcarriers (cytodex, HFP or CFP) were added to each well except for the no microcarrier control wells (negative control) and the positive tissue culture treated control wells (ThermoScientific). The plates were incubated at 37° C., 5% CO₂ and assessed at 24 hours or 72 hours for cell viability. Each well was prepared for the specific timepoint. AlamarBlue® Cell Viability Assay Reagent (Invitrogen) was used to quantify cellular metabolic activity and in turn determine the concentration of viable cells in a given sample. The dye incorporates an oxidation-reduction (REDOX) indicator that both fluoresces and change color in response to the chemical reduction due to cell growth. The higher the % reduction of media, the more metabolically active cells are present. In these experiments, if cells cultured with microcarriers had a higher percentage reduction than cells with no microcarrier, these were scored as viable because the cells have attached onto the microcarriers. The process is shown in FIG. 5.

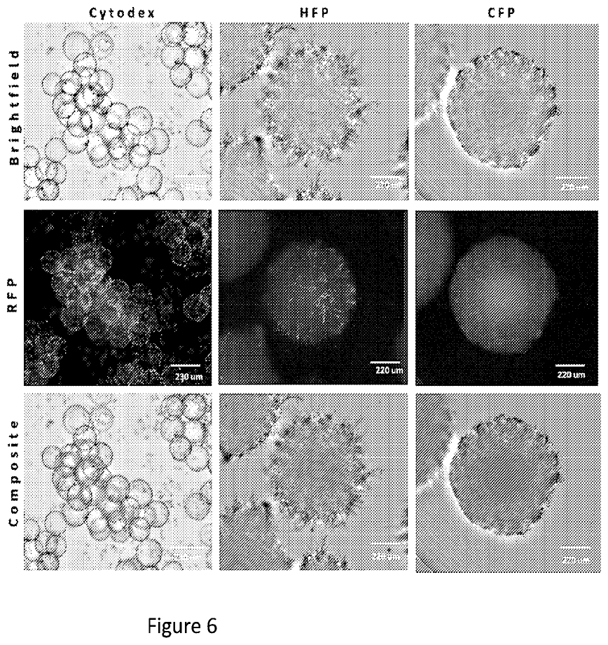

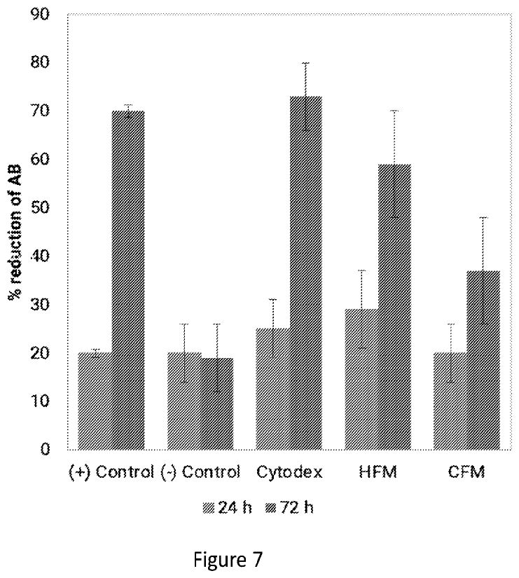

FIG. 6 shows images of brightfield, red fluorescent protein and composite images of each microcarrier. FIG. 7 shows a bar graph of cell viability as assessed by the AlamarBlue® Cell Viability Assay at the 24 h and 72 h time points for the negative and positive controls and the 3 microcarriers tested. The C2C12 cells remained viable on the CFP and HFP microcarriers, demonstrating that the fungal pellets provide a microcarrier structure compatible with animal cell attachment and viability. Furthermore, the % reduction increased from 24 h to 72 h, indicating that cells proliferated during the two time points when attached to HFP and CFP, with HFP showing higher proliferation than CFP.

### Example 4 Cell Seeding

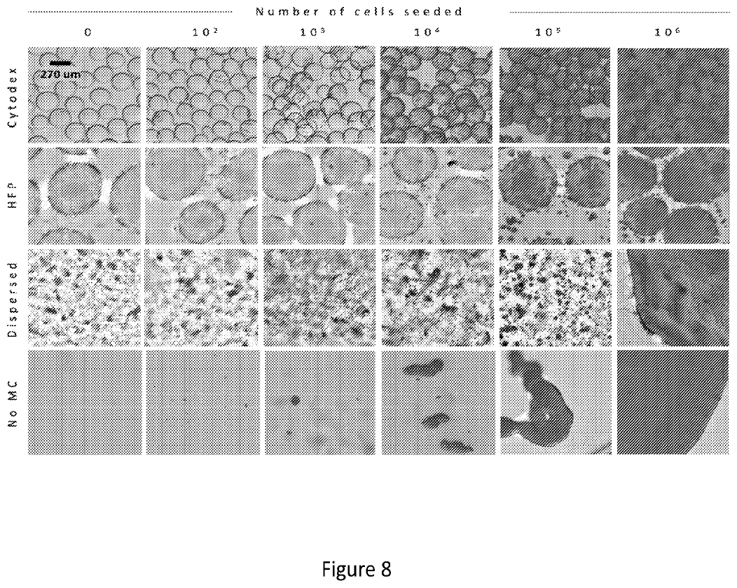

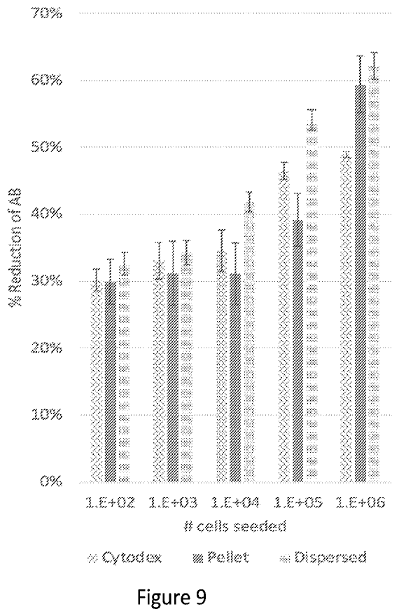

Following the method of Example 3 red fluorescent C2C12 cells transfected with tomato gene were seeded onto cytodex 3, HFP, dispersed hyphae or incubated without microcarrier at cell numbers between 0-10⁶ cells. Dispersed hyphae are HFP pellets that have been mechanically fragmented using a tissue homogenizer. This condition was to assess if the morphological shape of the fungal pellet impacted cell attachment and proliferation. Cells were grown for 72 days imaged using ImageXpress Pico Automated Cell Imaging System (FIG. 8) and viability assessed by AlamarBlue assay (FIG. 9). Results show that C2C12 cells attach and cover the HFP pellet at initial seeding densities of 10\hat{ }5 and 10\hat{ }6 cells. For dispersed pellet morphology, cells were distributed randomly on the fungal hyphae until seeding density at 10\hat{ }6 where cells started clumping together and forming aggregates. The aggregate formation was also seen in cytodex and no microcarrier conditions because the high concentration of cells forced them to self-adhere. Self-aggregate formation is typically not favorable for cell growth for this cell line and proliferation was not seen in 10\hat{ }6 seeding cell density for cytodex. However, dispersed hyphae growth showed proliferation even at 10\hat{ }6 seeding density despite aggregate formation.

### Example 6 Immobilization of Fish Cells

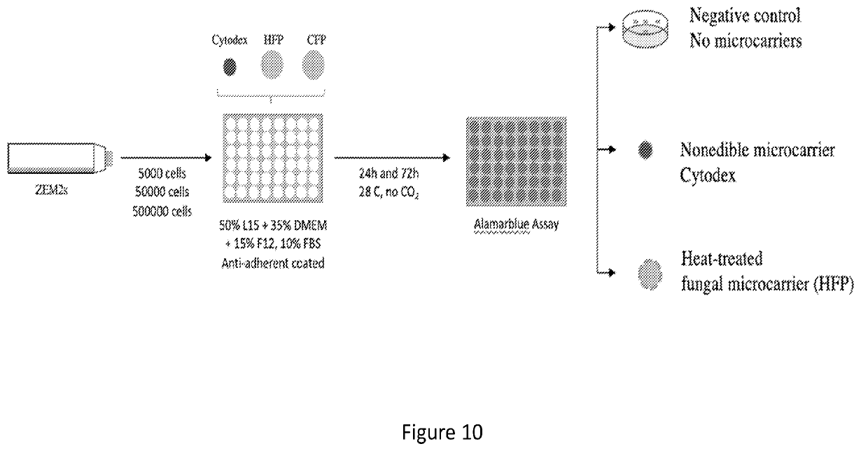

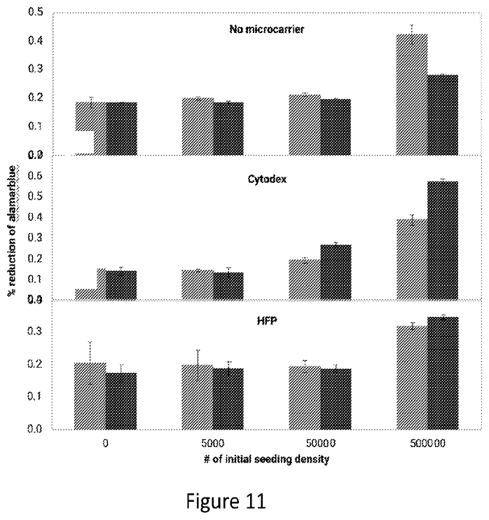

ZEM2S cells (Danio rerio, zebrafish; ATCC) were seeded onto multi-well anti-adherent coated plates at 5000 cells/well, 50,000 cells/well or 500,000 cells/well in media containing 50% L15+35% DMEM+15% F12 and 10% FBS. Microcarriers (cytodex and HFP (see Example 1) were added and the plates were incubated for 24 or 72 hours at 28° C. without CO₂. Following incubation, cell viability was analyzed by the AlamarBlue® Cell Viability Assay as described in Example 3. The process is shown in FIG. 10. Results are shown in FIG. 11. All microcarriers showed attachment and cell viability at the highest seeding density (500,000 cells) at the 24 h (light grey) and 72 hour (dark grey) timepoints.

### Example 7 Cell Surface Hydrophobicity

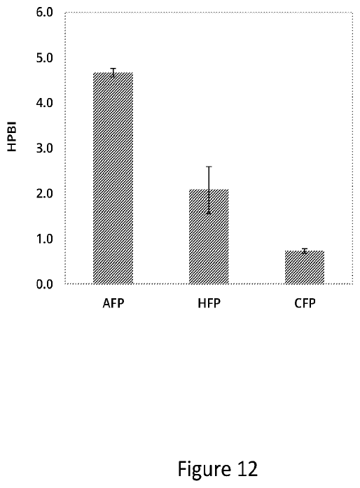

In order to assess the surface hydrophobicity of fungal pellet, a phase-partition method was used as described by Rosenberg et al. (1983). Active fungal pellets (AFP), heat treated pellets (HFP); and (c) chemically treated pellets (CFP) were prepared as described in Example 1. After washing with phosphate buffer saline (PBS), 0.5 ml of settled pellets was placed in PBS. Optical density of the cell suspension was measured at 640 nm (A1) using Genesys 10 s UV-Vis spectrophotometer (Thermo Scientific, Rochester, NY, USA). Then, 0.5 ml of octane (Alfa Aesar, Haverhill, MA, USA) was added into 3.5 ml of fungal suspension, then mixed with a vortex mixer for 2 minutes and allowed to stand for 10-15 minutes until phase separation was observed. The upper hydrocarbon phase was discarded, and the lower aqueous phase was collected and measured for optical density at 640 nm (A2). The hydrophobicity index (HPBI) was determined as: HPBI=A1−A2/A1*100. Results are shown in FIG. 12. AFP showed the highest HPBI, followed by HFP then CFP.

### Example 8 Fungal Pellet Comparison

Rhizopus oligosporus, Aspergillus sojae, Aspergillus awamori, Aspergillus oryzae FST 76-2, Aspergillus oryzae UCD8, Aspergillus tubingensis, Aspergillus nishimurae, and Penicillium chrysogenum fungal spores were inoculated into sporulation medium containing (1.7% (w/v) corn meal agar, 0.1% (w/v) yeast extract, 0.2% (w/v) glucose and 2% (w/v) agar) for 7 days at 28° C. Spores were suspended in sterile DI water, vortexed, sonicated for 5 minutes, and 1×10⁶ spore/mL final concentration inoculated in 50 mL of filamentous fungi pellet medium (6% (w/v) glucose, 0.3% (w/v) yeast extract, 0.3% (w/v) NaNO₃, 0.1% (w/v) K₂HPO₄, 0.05% (w/v) MgSO₄, 0.05% (w/v) KCl, 0.001% (w/v) FeSO4; pH adjusted to 5.5 with HCl) in 250 mL Erlenmeyer flasks covered with hydrophobic cotton. Cultures were shaken for 3 days at 30° C. until pellets were formed. Pellets were inactivated by heating to 121° C. for 20 minutes.

Measurements were taken of total wet weight of whole flask, wet weight per pellet, total dry weight of whole flask, dry weight per pellet, number of pellets per flask and pellet diameter. Results are shown in Table 3.

TABLE 3
Fungal Pellet Measurements

| | | | | A. | A. | | | |
|---|---|---|---|---|---|---|---|---|
| | R. | A. | A. | oryzae | oryzae | A. | A. | P. |
| | oligosporus | sojae | awamori | 1 | 2 | tubingensis | nishimurae | chrysogenum |
| Edible | Yes | Yes | Yes | Yes | Yes | No | No | No |
| Total wet | 3.12 ± | 11.80 ± | 14.5 ± | 5.91 ± | 8.61 ± | 9.73 ± | 9.00 ± | 23.69 ± 0.54 |
| weight whole | 0.96 | 3.82 | 7.23 | 0.73 | 0.93 | 1.02 | 2.43 | |
| flask (g) | | | | | | | | |
| Wet weight | n/a | 1.4 ± | 7.0 ± | 0.4 ± | 8.7 ± | 2.5 ± | 0.5 ± | 5.9 ± 0.7 |
| per pellet | | 0.4 | 3.8 | 0.1 | 3.6 | 0.7 | 0.2 | |
| (mg) | | | | | | | | |
| Total dry | 0.36 ± | 0.9 ± | 0.21 ± | 0.29 ± | 0.32 ± | 0.43 ± | 0.39 ± | 12.5 ± 0.14 |
| weight whole | 0.05 | 0.31 | 0.05 | 0.6 | 0.08 | 0.02 | 0.15 | |
| flask (g) | | | | | | | | |
| Dry weight | n/a | 0.17 ± | 0.3 ± | 0.03 ± | 0.3 ± | 0.18 ± | 0.05 ± | 0.3 ± 0.04 |
| per pellet | | 0.08 | 0.2 | 0.01 | 0.1 | 0.08 | 0.01 | |
| (mg) | | | | | | | | |
| Number | n/a | 6127 ± | 2515 ± | 16772 ± | 1128 ± | 4130 ± | 23014 ± | 4045 ± 483 |
| pellets per | | 1575 | 1501 | 4799 | 554 | 1342 | 14949 | |
| flask (#) | | | | | | | | |
| Diameter of | n/a | 1720 ± | 2990 ± | 1101 ± | 3050 ± | 2097 ± | 1200 ± | 2257 ± 71 |
| pellet (um) | | 427 | 650 | 17 | 580 | 162 | 78 | |

### Example 10 Viability of C2C12 Cells on Various Fungal Microcarriers

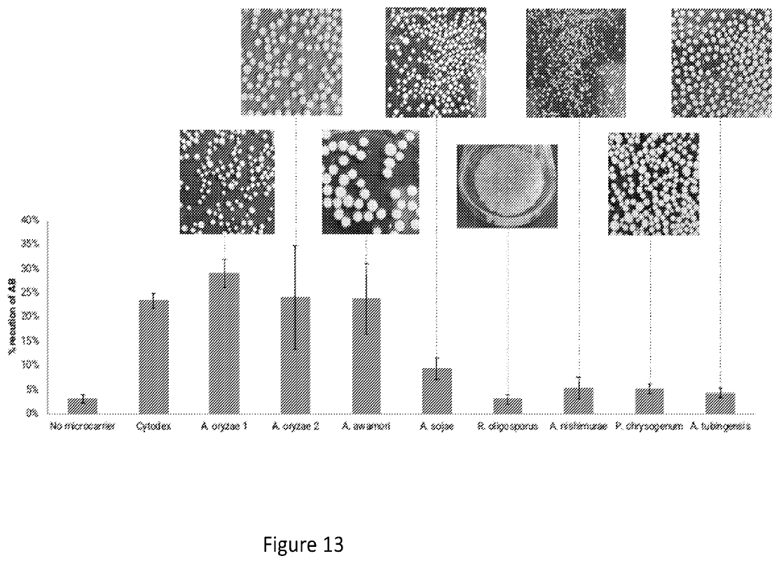

C2C12 cells were prepared as in Example 3 and seeded at 3000 cells/cm² onto anti-adherent coated (StemCell) multi-well plates (ThermoScientific) in 70% DMEM (Dulbecco's Modified Eagle Medium), media supplemented with 10% fetal bovine serum (FBS). Microcarriers (cytodex, HFP A. oryzae 76-2, HFP A. oryzae UCD 8, HFP A. awamori, HFP A. sojae, HFP R. oligosporus, HFP P. chysogenum, HFP A. tubingensis, HFP A. nishimurae, mechanically dispersed fungal microcarrier) were added to each well except for the no microcarrier control wells (negative control) and the positive tissue culture treated control wells (ThermoScientific). The plates were incubated at 37° C., 5% CO₂ and assessed at 24 hours or 72 hours. Following incubation, cell viability was analyzed by the AlamarBlue® Cell Viability Assay as described in Example 3. Results are shown in FIG. 13. FIG. 13 also shows images of HFP fungal microcarriers after 3 days cultivation.

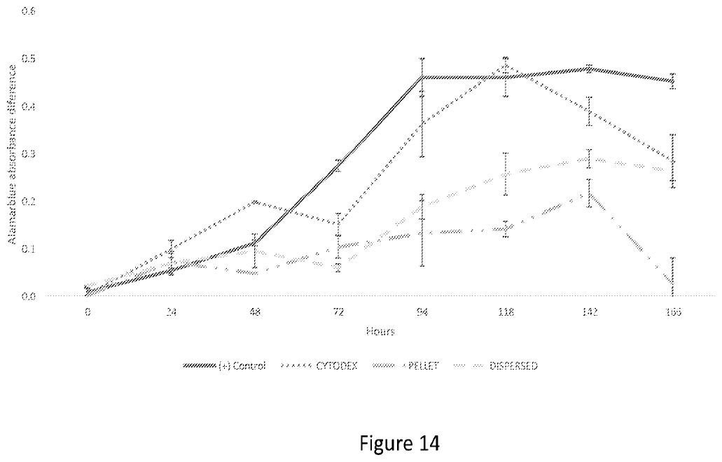

Viability of C2C12 cells over 7 days incubation seeded on cytodex, A. oryzae 76-2 pellet and A. oryzae 76-2 dispersed hyphae microcarriers is shown in FIG. 14. In these experiments, if cells cultured with microcarriers had a higher percentage reduction than cells with no microcarrier, these were scored as viable because the cells have attached onto the microcarriers.

### Example 10 Viability of bSC Cells after 3 Day Cultivation on A. oryzae

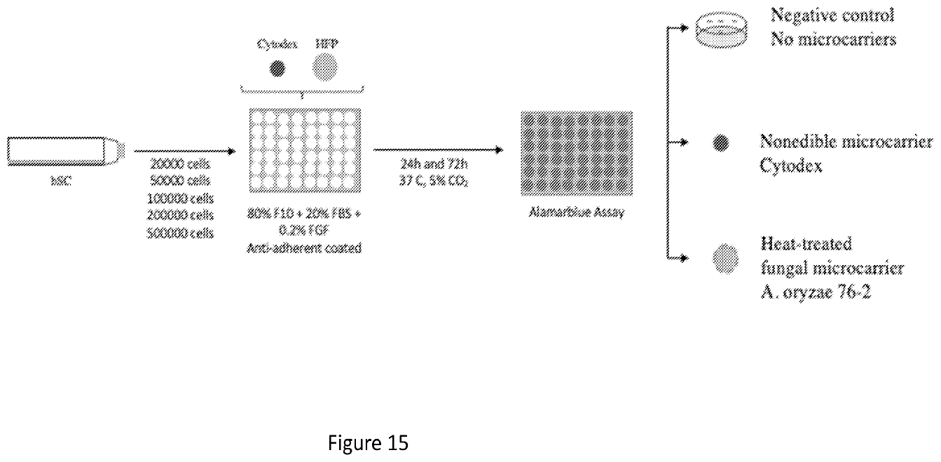

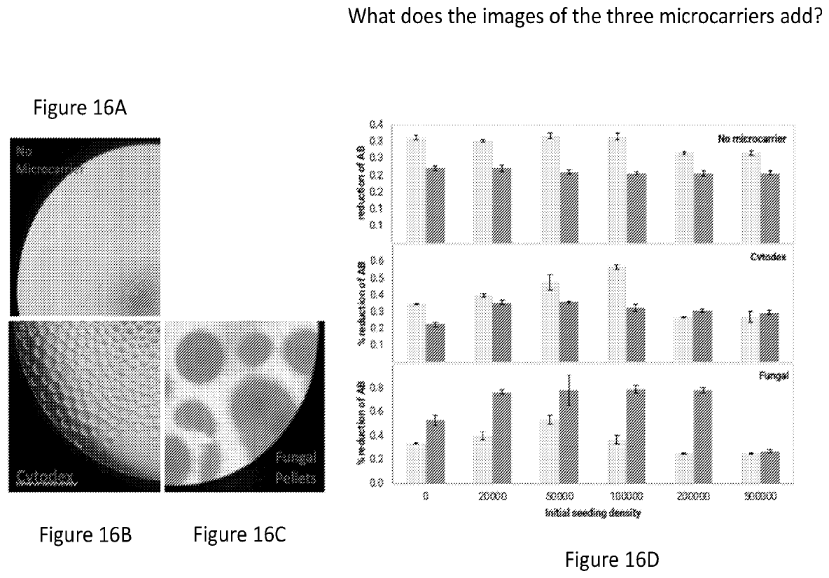

Bovine satellite cells (bSc) were seeded at either 20,000, 50,000, 100,000, 200,000, 500,000 cells/cm² onto anti-adherent coated (StemCell) multi-well plates (ThermoScientific) in 80% EM, media supplemented with 20% fetal bovine serum (FBS), and 0.2% fibroblast growth factor (FGF). Microcarriers (cytodex, HFP) were added to each well except for the no microcarrier control wells (negative control). The plates were incubated at 37° C., 5% CO₂. Following incubation, cell viability was analyzed and assessed for cell viability at 24 hours or 72 hours by the AlamarBlue® Cell Viability Assay as described in Example 3. Process is shown in FIG. 15. Images of the no microcarrier control is shown in FIG. 16A, the cytodex in FIG. 16B, and the fungal pellet in FIG. 16C. Viability of bovine satellite cells after 24 h (light grey bars) and 72 h (dark grey bars) on various microcarriers are shown in FIG. 16D.

### Example 11 Viability of bESC Cells on A. oryzae Microcarriers

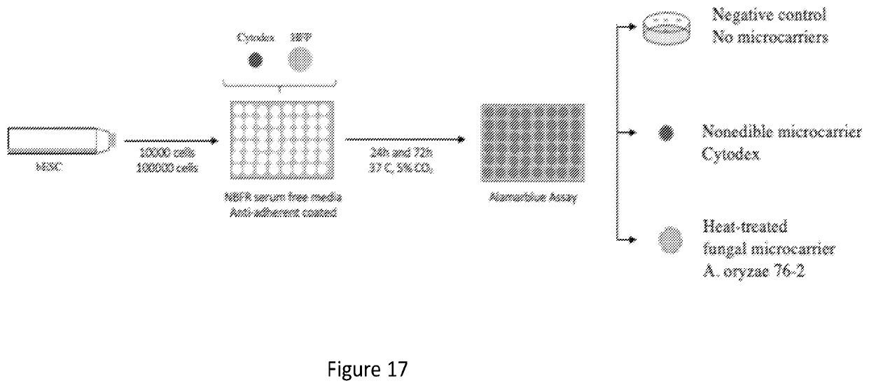

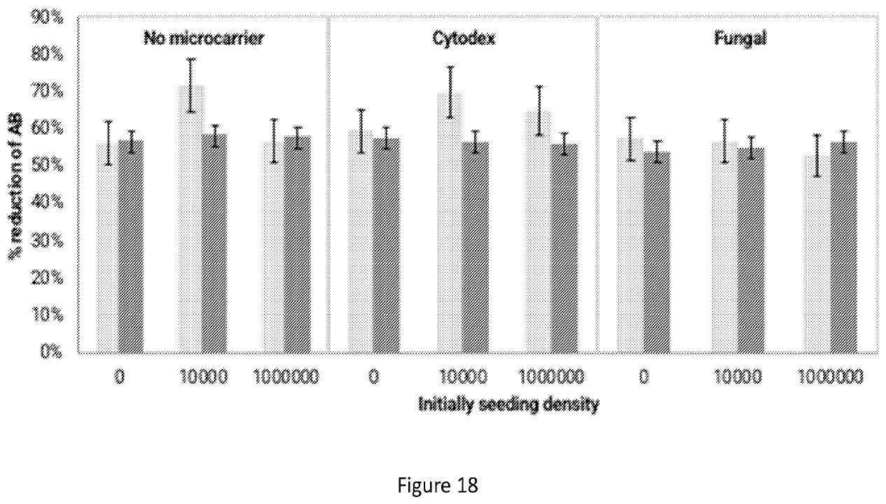

Bovine embryonic stem cells (bESC) were seeded at either 10,000 or 100,000 cells/cm² onto anti-adherent coated (StemCell) multi-well plates (ThermoScientific) in NBFR serum free media. Microcarriers (cytodex, HFP) were added to each well except for the no microcarrier control wells (negative control). The plates were incubated at 37° C., 5% CO₂. and for 24 hours or 72 hours. Following incubation, cell viability was analyzed by the AlamarBlue® Cell Viability Assay as described in Example 3. Process is shown in FIG. 17. Viability of bovine embryonic stem cells after 24 h (light grey bars) and 72 h (dark grey bars) on various microcarriers are shown in FIG. 18.

### Example 12 Differentiation of C2C12 Cells

Differentiation of C2C12 cells was assessed by detecting desmin, an early myogenic marker, by western blot. Four conditions were studied: differentiation media with no cells or pellets, fungal pellets only in differentiation media, C2C12 cells grown on tissue culture treated plates in differentiation media, and C2C12 cells grown on fungal pellets in differentiation media.

C2C12 cells were first grown on tissue culture plates or on A. oryzae 76-2 heat inactivated pellets in proliferation media (DMEM+10% FBS) (Thermo Scientific, Massachusetts) for 3 days. On the third day, proliferation media was replaced by differentiation media (DMEM+2% Horse Serum) (Thermo Scientific, Massachusetts) and cultivated for 3 days to induce differentiation. Sampling conditions were the following: Samples were extracted using RIPA Buffer (Thermo Scientific, Massachusetts) with 1 μM PMSF following Fortis Life Science protocol with slight modifications. Instead of using a probe tip sonicator, a sonication bath was used instead. 25 μL of lysate was combined with 8.33 μL of a 4:1 laemmli:β-ME solution (BioRad, California) and incubated at 95° C. for 5 min. After incubation, 30 μL of sample was loaded into each lane of the gel and allowed to run for 30 min at 200V. The gel was imaged and transferred to nitrocellulose membrane using Transblot system (Thermo Scientific, Massachusetts). Blocking was done by in 1×PBS (BioRad, California) and 1% casein solution (BioRad, California) for 1 hr at RT and further washed three times with PBST (BioRad, California). Incubation overnight was performed with DES primary antibody (DSHB, Iowa) at 4° C. and washed three times with PBST (BioRad, California) followed by another incubation in anti-mouse IgG secondary antibody solution (RD Minneapolis) for 1 hour at RT and washed three times with PBST. 1:1 mix of Clarity: Clarity Max ECL (BioRad, California) was added and gel was imaged on Chemidoc (BioRad, California).

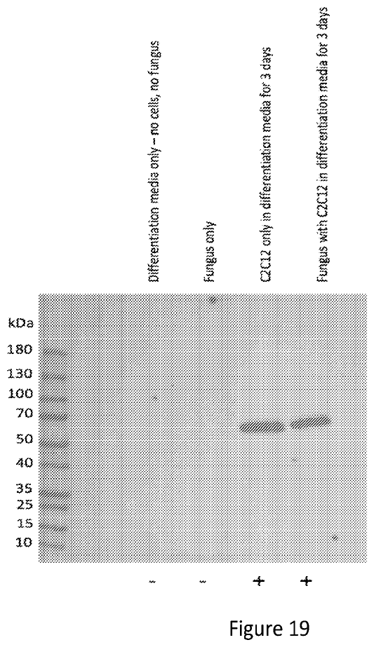

Results are shown in FIG. 19. Desmin was detected in C2C12 grown on tissue culture treated plates and fungal pellets (lanes 3 and 4, respectively), but not in the controls with only media or only fungal pellets (lanes 1 and 2, respectively). The desmin signal appeared similar in lanes 3 and 4, indicating that growth of the C2C12 on fungal pellets did not inhibit C2C12 capability to differentiate.

The following references may be relevant and or informative to the instant disclosure and are incorporated herein to the extent to which they do not conflict.
1. Derakhti S, Safiabadi-Tali S H, Amoabediny G, Sheikhpour M. Attachment and detachment strategies in microcarrier-based cell culture technology: A comprehensive review. Mater Sci Eng C Mater Biol Appl. 2019 October; 103:109782.
2. Oh S K W, Chen A K, Mok Y, Chen X, Lim U-M, Chin A, et al. Long-term microcarrier suspension cultures of human embryonic stem cells. Stem Cell Res. 2009 May; 2(3):219-30.
3. Rowley J, Abraham E, Campbell A, Brandwein H, Oh S. Meeting Lot-Size Challenges of Manufacturing Adherent Cells for Therapy. J BioProcess Int. 2012;
4. Moritz M S M, Verbruggen S E L, Post M J. Alternatives for large-scale production of cultured beef: A review. J Integr Agric. 2015 February; 14(2):208-16.
5. McKee C, Chaudhry G R. Advances and challenges in stem cell culture. Colloids Surf B Biointerfaces. 2017 Nov. 1; 159:62-77.
6. Lee S J, Yang S. Micro glass ball embedded gels to study cell mechanobiological responses to substrate curvatures. Rev Sci Instrum. 2012 September; 83(9):094302.
7. Baraniak P R, Cooke M T, Saeed R, Kinney M A, Fridley K M, McDevitt T C. Stiffening of human mesenchymal stem cell spheroid microenvironments induced by incorporation of gelatin microparticles. J Mech Behav Biomed Mater. 2012 July; 11:63-71.
8. Wang X, Wenk E, Zhang X, Meinel L, Vunjak-Novakovic G, Kaplan D L. Growth factor gradients via microsphere delivery in biopolymer scaffolds for osteochondral tissue engineering. J Control Release. 2009 Mar. 4; 134(2):81-90.
9. Zhang G, Zhao X, Li X, Du G, Zhou J, Chen J. Challenges and possibilities for bio-manufacturing cultured meat. Trends Food Sci Technol. 2020 January;
10. Bodiou V, Moutsatsou P, Post M J. Microcarriers for upscaling cultured meat production. Front Nutr. 2020 Feb. 20; 7:10.
11. Barbesgaard P, Heldt-Hansen H P, Diderichsen B. On the safety of Aspergillus oryzae: a review. Appl Microbiol Biotechnol. 1992 February; 36(5):569-72.
12. Berka R M, Nelson B A, Zaretsky E J, Yoder W T, Rey M W. Genomics of Fusarium venenatum: An Alternative Fungal Host for Making Enzymes. Fungal Genomics. Elsevier; 2004. p. 191-203.
13. Finnigan T J A, Wall B T, Wilde P J, Stephens F B, Taylor S L, Freedman M R. Mycoprotein: the future of nutritious nonmeat protein, a symposium review. Curr Dev Nutr. 2019 June; 3(6):nzz021.
14. Souza Filho P F, Andersson D, Ferreira J A, Taherzadeh M J. Mycoprotein: environmental impact and health aspects. World J Microbiol Biotechnol. 2019 Sep. 23; 35(10):147.
15. Meyer V, Basenko E Y, Benz J P, Braus G H, Caddick M X, Csukai M, et al. Growing a circular economy with fungal biotechnology: a white paper. Fungal Biol Biotechnol. 2020 Apr. 2; 7:5.
16. Veiter L, Rajamanickam V, Herwig C. The filamentous fungal pellet-relationship between morphology and productivity. Appl Microbiol Biotechnol. 2018 April; 102(7):2997-3006.
17. Xie S, Sun S, Dai S Y, S. Yuan J. Efficient coagulation of microalgae in cultures with filamentous fungi. Algal Research. 2013 January; 2(1):28-33.
18. Miranda A F, Taha M, Wrede D, Morrison P, Ball A S, Stevenson T, et al. Lipid production in association of filamentous fungi with genetically modified cyanobacterial cells. Biotechnol Biofuels. 2015 Nov. 5; 8:179.
19. Moreira M T, Sanromin A, Feijoo G, Lema J M. Control of pellet morphology of filamentous fungi in fluidized bed bioreactors by means of a pulsing flow. Application to Aspergillus niger and Phanerochaete chrysosporium. Enzyme Microb Technol. 1996 September; 19(4):261-6.
20. Moreno-Garcia J, Garcia-Martinez T, Moreno J, Mauricio J C, Ogawa M, Luong P, et al. Impact of Yeast Flocculation and Biofilm Formation on Yeast-Fungus Coadhesion in a Novel Immobilization System. Am J Enol Vitic. 2018 Jul. 1; 69(3):278-88.
21. Ogawa M, Bisson L F, Garcia-Martinez T, Mauricio J C, Moreno-Garcia J. New insights on yeast and filamentous fungus adhesion in a natural co-immobilization system: proposed advances and applications in wine industry. Appl Microbiol Biotechnol. 2019 June; 103(12):4723-31.
22. Zhang J, Zhang J. The filamentous fungal pellet and forces driving its formation. Crit Rev Biotechnol. 2016 December; 36(6):1066-77.
23. Li Z J, Shukla V, Fordyce A P, Pedersen A G, Wenger K S, Marten M R. Fungal morphology and fragmentation behavior in a fed-batch Aspergillus oryzae fermentation at the production scale. Biotechnol Bioeng. 2000 Nov. 5; 70(3):300-12.
24. Schmidt J J, Jeong J, Kong H. The interplay between cell adhesion cues and curvature of cell adherent alginate microgels in multipotent stem cell culture. Tissue Eng Part A. 2011 November; 17(21-22):2687-94.
25. Werner M, Blanquer S B G, Haimi S P, Korus G, Dunlop J W C, Duda G N, et al. Surface curvature differentially regulates stem cell migration and differentiation via altered attachment morphology and nuclear deformation. Adv Sci (Weinh). 2017; 4(2):1600347.
26. Werner M, Petersen A, Kurniawan N A, Bouten C V C. Cell-Perceived Substrate Curvature Dynamically Coordinates the Direction, Speed, and Persistence of Stromal Cell Migration. Adv Biosys. 2019 Sep. 5; 3(10):e1900080.
27. Saeedi Garakani S, Khanmohammadi M, Atoufi Z, Kamrava S K, Setayeshmehr M, Alizadeh R, et al. Fabrication of chitosan/agarose scaffolds containing extracellular matrix for tissue engineering applications. Int J Biol Macromol. 2020 Jan. 15; 143:533-45.
28. Kubis H-P, Scheibe R J, Decker B, Hufendiek K, Hanke N, Gros G, et al. Primary skeletal muscle cells cultured on gelatin bead microcarriers develop structural and biochemical features characteristic of adult skeletal muscle. Cell Biol Int. 2016 April; 40(4):364-74.
29. Tronchin G, Pihet M, Lopes-Bezerra L M, Bouchara J-P. Adherence mechanisms in human pathogenic fungi. Med Mycol. 2008 December; 46(8):749-72.
30. He J, Genetos D C, Leach J K. Osteogenesis and trophic factor secretion are influenced by the composition of hydroxyapatite/poly(lactide-co-glycolide) composite scaffolds. Tissue Eng Part A. 2010 January; 16(1):127-37.
31. Verbruggen S, Luining D, van Essen A, Post M J. Bovine myoblast cell production in a microcarriers-based system. Cytotechnology. 2018 April; 70(2):503-12.
32. Chan L G, Dias filamentous fungusG, Saarni A, Cohen J, Block D, Taha A Y, et al. Scaling up the Bioconversion of Cheese Whey Permeate into Fungal Oil by Mucor circinelloides. J Am Oil Chem Soc. 2020 July; 97(7):703-16.
33. Oddone G M, Lan C Q, Rawsthorne H, Mills D A, Block D E. Optimization of fed-batch production of the model recombinant protein GFP in Lactococcus lactis. Biotechnol Bioeng. 2007 Apr. 15; 96(6):1127-38.
34. Verstrepen K J, Klis F M (2006) Flocculation, adhesion and biofilm formation in yeasts. Mol Microbiol 60:5-15. https://doi.org/10.1111/j.1365-2958.2006.05072.x Soares E V (2011) Flocculation in Saccharomyces cerevisiae: a review. J Appl Microbiol 110:1-18. https://doi.org/10.1111/j.1365-2672.2010.04897
35. Di Gianvito P, Tesniere C, Suzzi G, Blondin B, Tofalo R (2017) FLO 5 gene controls flocculation phenotype and adhesive properties in a Saccharomyces cerevisiae sparkling wine strain. Sci Rep 7:10786. https://doi.org/10.1038/s41598-017-09990-9
36. Verstrepen K J, Derdelinckx G, Verachtert H, Delvaux F R (2003) Yeast flocculation: what brewers should know. Appl Microbiol Biotechnol 61:197-205. https://doi.org/10.1007/s00253-002-1200-8
37. Miki B L, Poon N H, Seligy V L (1982) Repression and induction of flocculation interactions in Saccharomyces cerevisiae. J Bacteriol 150:890-899
38. Stratford M (1989) Yeast flocculation: calcium specificity. Yeast 5:487-496. https://doi.org/10.1002/yea.320050608
39. Mezu-Ndubuisi O J, Maheshwari A. The role of integrins in inflammation and angiogenesis. Pediatr Res. 2021 89, 1619-1626. https://doi.org/10.1038/s41390-020-01177-9
40. Cerqueira G C, Arnaud M B, Inglis D O, Skrzypek M S, Binkley G, Simison M, Miyasato S R, Binkley J, Orvis J, Shah P, Wymore F, Sherlock G, Wortman J R. The Aspergillus Genome Database: multispecies curation and incorporation of RNA-Seq data to improve structural gene annotations. Nucleic Acids Res. 2014 42 (1); D705-10.
41. Bodiou V, Moutsatsou P, Post M J. Microcarriers for upscaling cultured meat production. Front Nutr. 2020 Feb. 20; 7:10.
42. Mo X M, Xu C Y, Kotaki M, Ramakrishna S. Electrospun P(LLACL) nanofiber: a biomimetic extracellular matrix for smooth muscle cell and endothelial cell proliferation. Biomaterials. 2004 25:1883-90. doi: 10.1016/j.biomaterials.2003.08.042

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims

1. A composition comprising a fungal pellet and animal cells, wherein the animal cells are in connection with the fungal pellet, wherein the fungal pellet is inviable.

2. The composition of claim 1, wherein the animal cells or a portion thereof are located on an exterior surface of the fungal pellet.

3. The composition of any one of claims 1-2, wherein the animal cells or a portion thereof are located in an interior of the fungal pellet.

4. The composition of any one of claims 1-3, wherein the fungal pellet comprises intact fungal cells.

5. The composition of any one of claims 1-4, wherein the fungal pellet comprises a filamentous fungus.

6. The composition of any one of claims 1-5, wherein the fungal pellet has been heat-treated, chemically treated, or lyophilized.

7. The composition of any one of claims 1-6, wherein the fungal pellet comprises a fungus selected from the group consisting of Rhizopus, Aspergillus, Penicillium, and combinations thereof.

8. The composition of any one of claims 1-6, wherein the fungal pellet comprises a fungus selected from the group consisting of Rhizopus oligosporus, Aspergillus sojae, Aspergillus awamori, Aspergillus oryzae, Aspergillus tubingensis, Aspergillus nishimurae, Penicillium chrysogenum, and combinations thereof.

9. The composition of any one of claims 1-8, wherein the animal cells are mammalian cells.

10. The composition of claim 9, wherein the mammalian cells are non-human mammalian cells.

11. The composition of any one of claims 1-8, wherein the animal cells are fish cells.

12. The composition of any one of claims 1-10, wherein the animal cells are cow, pig, chicken, goat, and/or sheep.

13. The composition of any one of claims 1-12, wherein the composition is Generally Recognized as Safe (GRAS).

14. The composition of any one of claims 1-13, wherein the fungal pellet has a diameter of 0.8 mm to 1.1 mm.

15. The composition of any one of claims 1-14, wherein the volume of the fungal pellet is between 0.013 mm³ to 0.018 mm³.

16. A food product comprising the composition of any one of claims 1-15.

17. A process for producing a composition of animal cells, comprising:
a. obtaining a fungal pellet comprising a fungus, wherein the fungus is inviable or inactivated;
b. seeding a plurality of animal cells, which are viable, onto the fungal pellet to produce a seeded fungal pellet; and
c. incubating the seeded fungal pellet under conditions wherein the animal cells remain viable.

18. The process of claim 17, further comprising harvesting the seeded fungal pellet after incubating.

19. The process of claim 17 or claim 18, wherein the fungal pellet is obtained by inoculating fungal spores into a liquid growth medium, wherein after an incubation period of at least 2 hours at a temperature between 15° C. to 45° C., the fungal spores form a fungal pellet comprising mycelium.

20. The process of any one of claims 17-19, wherein the fungal pellet comprises a filamentous fungus.

21. The process of any one of claims 17-20, wherein the fungal pellet comprise a fungus selected from the group consisting of Rhizopus, Aspergillus, Penicillium, and combinations thereof.

22. The process of any one of claims 17-20, wherein the fungal pellet comprise a fungus selected from the group consisting of Rhizopus oligosporus, Aspergillus sojae, Aspergillus awamori, Aspergillus oryzae, Aspergillus tubingensis, Aspergillus nishimurae, Penicillium chrysogenum, and combinations thereof.

23. The process of any one of claims 17-22, wherein the fungus is inviable or inactivated by heat-treating or chemically treating the fungal pellet.

24. The process of any one of claims 17-23, wherein the animal cells are mammalian cells.

25. The process of claim 24, wherein the mammalian cells are non-human mammalian cells.

26. The process of claim 24, wherein the mammalian cells are cow, pig, chicken, goat, sheep, or combinations thereof.

27. The process of any one of claims 17-23, wherein the animal cells are fish cells.

28. The process of any one of claims 17-27, wherein the animal cells or a portion thereof are located on an exterior surface of the fungal pellet.

29. The process of any one of claims 17-28, wherein the fungal pellet is heat treated for 15-30 minutes at a temperature between 110-130° C.

30. The process of any one of claims 17-28, wherein the fungal pellet is chemically treated with 70% ethanol for 15-30 minutes.

31. An edible composition made by the processing according to any of claims 17-30.
